(19) United States
(12) Patent Application Publication
(10) Pub. No.: US 2023/0310409 A1
(43) Pub. Date: Oct. 5, 2023

(54) TREATING RHEUMATOID ARTHRITIS

(71) Applicant: BRISTOL-MYERS SQUIBB COMPANY, Princeton, NJ (US)

(72) Inventors: Gary L. Schieven, Lawrenceville, NJ (US); Shailesh Dudhgaonkar, Bangalore (IN); Michael Louis Doyle, Yardley, PA (US)

(21) Appl. No.: 18/011,884

(22) PCT Filed: Jun. 21, 2021
(86) PCT No.: PCT/US2021/038170

§ 371 Date: Dec. 21, 2022

(30) Foreign Application Priority Data
Jun. 22, 2020 (IN) 202011026256

(51) Int. Cl. A61K 31/4545; A61K 38/17; A61K 39/395; C07K 14/715; A61P 19/02

(52) U.S. Cl. CPC A61K 31/4545; A61K 38/1793; A61P 19/02; C07K 14/7151; A61K 39/3955

(57) ABSTRACT

Disclosed is a method of treating rheumatoid arthritis, comprising administering to a patient a therapeutically effective dose of a TLR7/8 inhibitor or a pharmaceutically acceptable salt thereof, in combination with a therapeutically effective dose of a TNFα inhibitor.

### CROSS REFERENCE

This application claims the benefit of Indian Provisional Application No. 202011026256 filed Jun. 22, 2020 which is incorporated herein in its entirety.

### DESCRIPTION

The present invention generally relates a method of treating a patient having rheumatoid arthritis, comprising administering to said patient a therapeutically effective dose of a TLR7/8 inhibitor or a pharmaceutically acceptable salt thereof, in combination with a therapeutically effective dose of a TNFα inhibitor.

### BACKGROUND OF THE INVENTION

TLR7 and TLR8 are endosomal receptors that recognize short uracil (U)-rich single strand RNA (ssRNA) (Junt J and Barchet W., Nat Rev Immunol. 2015; 15:529-544). TLR7 is expressed in plasmacytoid dendritic cells (pDC) and B cells. TLR7 agonists induce B cell activation and cytokine production, as well as IFNα production by pDC (Marshak-Rothstein A and Rifkin I R., Ann Rev Immunol. 2007; 25:419-41; Celhar T, Magalhaes R and Fairhurst A M., Immunol Res. 2012; 53:58-77). TLR8 is expressed in myeloid dendritic cells (mDC) and induces expression of cytokines such as IL-6, TNFα, and IL-10 (Gorden K B, Gorski K S, Gibson S J et al., J Immunol. 2005; 174:1259-1268; Cervantes J L, Weinerman B, Basole C. et al., Cell Mol Immunol. 2012; 9:434-438). TLR8 also induces expression of important cell surface molecules involved in antigen presenting cell interactions with T cells including CD40 and CD86, as well as other markers such as CD319 (SLAMF7).

TLR7 acts on pDC in an IFN-independent manner to induce high levels of resistance to glucocorticoids (Guiducci C, Gong M, Xu Z et al., Nature 2010; 465:937-941). TLR7 activates the NF-kB pathway in pDC, driving responses including expression of Bcl-2 leading to increased pDC survival. Glucocorticoids do not affect NF-kB activation in pDC. This blocks the ability of glucocorticoids to inhibit IFN production by pDC and also induces strong protection against glucocorticoid induced apoptosis. TLR7 stimulation of B cells induces glucocorticoid resistance by the cells, inhibiting the ability of glucocorticoids to inhibit B cell responses and induce apoptosis. The induction of glucocorticoid resistance is believed to be the reason treatment of systemic lupus erythematosus (SLE) requires much higher glucocorticoid doses than many other autoimmune diseases.

TLR7 and 8 are normally activated by pathogen associated RNA, and can also be activated by synthetic small molecule agonists. However, they are activated by self-RNA as part of the disease pathophysiology of SLE and related autoimmune diseases such as Sjögren's Syndrome (Celhar T, Magalhaes R and Fairhurst A M., Immunol Res. 2012; 53:58-77; Celhar T and Fairhurst A M., Frontier Pharm. 2014; 5:1-8). Activated TLR7 and 8 drive multiple responses across cell types that drive disease pathophysiology in lupus, forming a cycle of disease that acts as a feed-forward loop to accelerate disease progression (Davidson A and Aranow C., Nat Rev Rheum. 2010; 6:13-20). TLR7 stimulation of B cells induces B cell activation, production of proinflammatory cytokines, and is required for the formation of spontaneous germinal centers that are involved in the generation of high affinity autoantibodies involved in SLE. This applies to antibodies to many auto-antigens, not only RNA associated antigens. The increased production of autoantibodies leads to increased immune complex formation that in turn delivers increasing TLR7 and 8 stimulation, driving the disease cycle more and more strongly leading to disease progression.

Rheumatoid arthritis (RA) is a chronic autoimmune inflammatory disease that affects 1% of the population. Disease progression is characterized by a destructive inflammation of the joints, which can lead to progressive disability and a reduced life expectancy. The synovial membrane in RA is infiltrated by activated immune cells, most abundantly macrophages and T cells, resulting in the chronic production of proinflammatory cytokines and matrix metalloproteinases, leading to inflammation and cartilage and bone degradation (Choy E H and Panayi G S., N Engl J Med. 2001; 344:907-916).

Dysregulated TLR signaling has been implicated in several autoimmune and inflammatory diseases including RA where TLRs are important mediators of chronic inflammation especially in synovium (Thwaites R, Chamberlain G, and Sacre S., Front Immunol. 2014; 5:1). It has been reported that expression of several TLRs including endosomal TLRs are higher in RA synovial tissue as compared to tissue derived from either healthy controls or osteoarthritis patients. Components of necrotic cells and damaged tissues such as nucleic acid binding proteins, heat shock proteins, and extracellular matrix proteins have been shown to activate TLRs resulting in upregulation of cytokines and chemokines. Published reports on TLR7 knock-out mice and selective TLR7 and 9 antagonists to elucidate the role of TLRs in RA disease models support the use of TLR7 in the treatment of rheumatoid arthritis. Furthermore, human TLR8 activation in the joints promotes spontaneous and induced arthritis in mice. Together these studies indicate that TLR7 and TLR8 play a key role in RA and suggest that targeting TLR7 and/or TLR8 with antagonists may provide a new strategy for treatment of RA (Thwaites R, Chamberlain G, and Sacre S., Front Immunol. 2014; 5:1; Alzabin S, Kong P, Medghalchi M, et al., Arthritis Res Ther. 2012; 14: R142; Hoffmann M H, Skriner K, Herman S et al. Autoimmun. 2011; 36:288; Hayashi T, Gray C S, Chan M et al. Proc Natl Acad Sci USA. 2009; 106:2764; Guiducci C, Gong M, Cepika A M, et al. J Exp Med. 2013; 210:2903).

New methods of treating rheumatoid arthritis are desired.

Disclosed herein is a method of treating rheumatoid arthritis, comprising administering to a patient a therapeutically effective dose of a TLR7/8 inhibitor or a pharmaceutically acceptable salt thereof, in combination with a therapeutically effective dose of a TNFα inhibitor.

### SUMMARY OF THE INVENTION

The present invention provides a method of treating rheumatoid arthritis, comprising administering to a patient a therapeutically effective dose of a TLR7/8 inhibitor or a pharmaceutically acceptable salt thereof, in combination with a therapeutically effective dose of a TNFα inhibitor.

The present invention provides a method of treating rheumatoid arthritis, comprising administering to a patient a therapeutically effective dose of a TLR7 inhibitor or a pharmaceutically acceptable salt thereof, in combination with a therapeutically effective dose of a TNFα inhibitor.

The present invention provides a method of treating rheumatoid arthritis, comprising administering to a patient a therapeutically effective dose of a TLR8 inhibitor or a pharmaceutically acceptable salt thereof, in combination with a therapeutically effective dose of a TNFα inhibitor.

These and other features of the invention will be set forth in expanded form as the disclosure continues.

### BRIEF DESCRIPTION OF THE DRAWINGS

The invention is illustrated by reference to the accompanying drawings described below.

FIG. 1A and FIG. 1B show the inhibition of disease activity in the collagen-induced arthritis model by Compound (I) alone and in combination with mEnbrel.

FIG. 2A and FIG. 2B show inhibition of anti-collagen antibodies and IL-6, respectively, by Compound (I) alone and in combination with mEnbrel.

FIG. 3 shows the pharmacokinetics of Compound (I) and in combination with mEnbrel in a collagen-induced arthritis model.

### DEFINITIONS

In order that the present description may be more readily understood, certain terms are first defined. Additional definitions are set forth throughout the detailed description.

A “TLR7 inhibitor” inhibits the function of TLR7. TLR7 inhibitors can associate with TLR7 reversibly or irreversibly, and include antibodies, oligonucleotides, small molecules, and millimolecular compounds.

A “TLR8 inhibitor” inhibits the function of TLR8. TLR8 inhibitors can associate with TLR8 reversibly or irreversibly, and include antibodies, small molecules, and millimolecular compounds.

A “TLR7/8 inhibitor” inhibits the function of TLR7, TLR8, or both TLR7 and TLR8. TLR7/8 inhibitors can associate with TLR7 and TLR8 reversibly or irreversibly, and include antibodies, small molecules, and millimolecular compounds.

The compound of Formula (I) is a TLR7/8 inhibitor and has the structure:

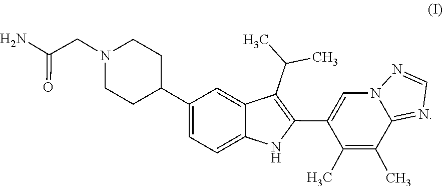 [SMILES: Cc1c(-c2[nH]c3ccc(C4CCN(CC(N)=O)CC4)cc3c2C(C)C)cn2ncnc2c1C]

The chemical name for the compound of Formula (I) is 2-(4-(2-(7,8-dimethyl-[1,2,4]triazolo[1,5-a]pyridin-6-yl)-3-isopropyl-1H-indol-5-yl)piperidin-1-yl)acetamide. The discovery and synthesis of the compound of Formula (I) is described in WO 2018/005586 A1.

A “TNFα inhibitor” is a drug that blocks the activity of tumor necrosis factor α (TNFα), and includes antibodies, small molecules, and millimolecular compounds. TNFα inhibitors include, but are not limited to, etanercept (Enbrel®), infliximab (Remicade®), certolizumab (Cimzia®), golimumab (Simponi®), adalimumab (Humira®), and biosimilars such as adalimumab-adbm (Cyltezo®), adalimumab-adaz (Hyrimoz®), adalimumab-atto (Amjevita®), etanercept-szzs (Erelzi®), infliximab-abda (Renflexis®), and infliximab-dyyb (Inflectra®).

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

The terms “treat,” “treating,” and “treatment,” as used herein, refer to any type of intervention or process performed on, or administering an active agent to, the patient with the objective of reversing, alleviating, ameliorating, inhibiting, or slowing down or preventing the progression, development, severity or recurrence of a symptom, complication, condition or biochemical indicia associated with a disease. Treatment includes therapeutic treatment and prophylactic or preventative measures, wherein the object is prevent or lessen the targeted condition or disorder.

The term “therapeutically effective amount” or “therapeutically effective dosage” of a drug or therapeutic agent refers to an amount of a drug effective to treat a disease or disorder in a patient. In certain embodiments, an effective amount refers to an amount effective, at dosages and for period of time necessary, achieve the desired therapeutic or prophylactic result. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Therapeutically effective amounts of a TLR7/8 inhibitor may vary according to factors such as the disease state, age, sex, and weight of the patient, and abilities of the TLR7/8 inhibitor to elicit a desired response in the patient. Therapeutically effective amounts of the TLR7/8 inhibitor encompasses an amount in which any toxic or detrimental effects of the TLR7/8 inhibitor are outweighed by the therapeutically beneficial effects.

The terms “administering” and “administration” refers to the physical introduction of a composition comprising a therapeutic agent to a patient, using any of the various methods and delivery systems known to those skilled in the art. Routes of administration for the TLR7/8 inhibitor and the TNFα inhibitor include enteral, topical, and mucosal administration such as oral, topical, sublingual, rectal, intranasal, and intravenous administration, and parenteral administration such as intravenous, intramuscular, and subcutaneous injection.

Administration “in combination with” one or more further therapeutic agents includes simultaneous (concurrent) and consecutive (sequential) administration in any order. For example, the patient may swallow the oral dosage form of the TLR7/8 inhibitor and the oral dosage form for the second agent in either order (consecutive); or may swallow both oral dosage forms together (concurrent).

The term “patient” includes human and other mammalian subjects that receive therapeutic treatment.

### DETAILED DESCRIPTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Provided herein are one or more methods of treating a patient having rheumatoid arthritis.

One embodiment provides a method of treating rheumatoid arthritis, comprising administering to a patient a therapeutically effective dose of a TLR7/8 inhibitor or a pharmaceutically acceptable salt thereof, in combination with a therapeutically effective dose of a TNFα inhibitor. Included in this embodiment is a method in which said TLR7/8 inhibitor is the compound of Formula (I).

One embodiment provides a method of treating rheumatoid arthritis, comprising administering to a patient a therapeutically effective dose of a TLR7 inhibitor or a pharmaceutically acceptable salt thereof, in combination with a therapeutically effective dose of a TNFα inhibitor. Included in this embodiment is a method in which said TLR7/8 inhibitor is the compound of Formula (I).

One embodiment provides a method of treating rheumatoid arthritis, comprising administering to a patient a therapeutically effective dose of a TLR8 inhibitor or a pharmaceutically acceptable salt thereof, in combination with a therapeutically effective dose of a TNFα inhibitor. Included in this embodiment is a method in which said TLR7/8 inhibitor is the compound of Formula (I).

One embodiment provides a method of treating rheumatoid arthritis, comprising administering to a patient a therapeutically effective dose of a TLR7/8 inhibitor or a pharmaceutically acceptable salt thereof, in combination with a therapeutically effective dose of a TNFα inhibitor, wherein said TNFα inhibitor is administered simultaneously with said TLR7/8 inhibitor. Included in this embodiment is a method in which said TLR7 inhibitor is the compound of Formula (I). Included in this embodiment is a method in which said TLR7/8 inhibitor is the compound of Formula (I).

One embodiment provides a method of treating rheumatoid arthritis, comprising administering to a patient a therapeutically effective dose of a TLR7/8 inhibitor or a pharmaceutically acceptable salt thereof, in combination with a therapeutically effective dose of a TNFα inhibitor, wherein said TNFα inhibitor is administered sequentially with said TLR7/8 inhibitor. Included in this embodiment is a method in which said TLR7/8 inhibitor is administered prior to the administration of said TNFα inhibitor. Also included in this embodiment is a method in which said TLR7/8 inhibitor is administered after said TNFα inhibitor. Additionally, included in this embodiment is a method in which said TLR7/8 inhibitor is the compound of Formula (I).

In one embodiment, a therapeutically effective dose of the compound of Formula (I) is in the range of 0.1 to 100 mg.

The therapeutically effective dose of the TL7/8 inhibitor can be administered as a single daily dose (q.d.), divided and administered twice daily (b.i.d.), or divided and administered as three or more doses per day.

The therapeutically effective dose of the TNFα inhibitor can be administered as prescribed in the dosing and administration instructions. The TNFα inhibitor can be administered as an infusion or as a subcutaneous injection. Dosing schedules include once every 1 to 8 weeks.

In one embodiment, a method is provided wherein the therapeutically effective dose of said TLR7/8 inhibitor is administered as a single daily dose.

In one embodiment, a method is provided wherein the therapeutically effective dose of the compound of Formula (I) is administered as a single daily dose.

In one embodiment, a method is provided wherein the therapeutically effective dose of said TNFα inhibitor is administered once per week.

In one embodiment, a method is provided wherein the therapeutically effective dose of said TNFα inhibitor is administered once every two weeks.

In one embodiment, a method is provided wherein the therapeutically effective dose of said TNFα inhibitor is administered once every three weeks.

In one embodiment, a method is provided wherein the therapeutically effective dose of said TNFα inhibitor is administered once every four weeks.

In one embodiment, a method is provided wherein the therapeutically effective dose of said TNFα inhibitor is administered once every five weeks.

In one embodiment, a method is provided wherein the therapeutically effective dose of said TNFα inhibitor is administered once every six weeks.

In one embodiment, a method is provided wherein the therapeutically effective dose of said TNFα inhibitor is administered once every seven weeks.

In one embodiment, a method is provided wherein the therapeutically effective dose of said TNFα inhibitor is administered once every eight weeks.

In one embodiment, a method is provided wherein the therapeutically effective dose of said TLR7/8 inhibitor is administered as a single daily dose; and said TNFα inhibitor is administered once every week. Included in this embodiment is a method in which said TLR7/8 inhibitor is the compound of Formula (I). Also included in this embodiment is a method in which said TNFα inhibitor is etanercept.

In one embodiment, a method is provided wherein the therapeutically effective dose of said TLR7/8 inhibitor is administered as a single daily dose; and said TNFα inhibitor is administered once every two weeks. Included in this embodiment is a method in which said TLR7/8 inhibitor is the compound of Formula (I). Also included in this embodiment is a method in which said TNFα inhibitor is etanercept.

In one embodiment, a method is provided wherein the therapeutically effective dose of said TLR7/8 inhibitor is administered as a single daily dose; and said TNFα inhibitor is administered once every three weeks. Included in this embodiment is a method in which said TLR7/8 inhibitor is the compound of Formula (I). Also included in this embodiment is a method in which said TNFα inhibitor is etanercept.

In one embodiment, a method is provided wherein the therapeutically effective dose of said TLR7/8 inhibitor is administered as a single daily dose; and said TNFα inhibitor is administered once every four weeks. Included in this embodiment is a method in which said TLR7/8 inhibitor is the compound of Formula (I). Also included in this embodiment is a method in which said TNFα inhibitor is etanercept.

In one embodiment, a method is provided wherein the therapeutically effective dose of said TLR7/8 inhibitor is administered as a single daily dose; and said TNFα inhibitor is administered once every five weeks. Included in this embodiment is a method in which said TLR7/8 inhibitor is the compound of Formula (I). Also included in this embodiment is a method in which said TNFα inhibitor is etanercept.

In one embodiment, a method is provided wherein the therapeutically effective dose of said TLR7/8 inhibitor is administered as a single daily dose; and said TNFα inhibitor is administered once every six weeks. Included in this embodiment is a method in which said TLR7/8 inhibitor is the compound of Formula (I). Also included in this embodiment is a method in which said TNFα inhibitor is etanercept.

In one embodiment, a method is provided wherein the therapeutically effective dose of said TLR7/8 inhibitor is administered as a single daily dose; and said TNFα inhibitor is administered once every seven weeks. Included in this embodiment is a method in which said TLR7/8 inhibitor is the compound of Formula (I). Also included in this embodiment is a method in which said TNFα inhibitor is etanercept.

In one embodiment, a method is provided wherein the therapeutically effective dose of said TLR7/8 inhibitor is administered as a single daily dose; and said TNFα inhibitor is administered once every eight weeks. Included in this embodiment is a method in which said TLR7/8 inhibitor is the compound of Formula (I). Also included in this embodiment is a method in which said TNFα inhibitor is etanercept.

Another embodiment provides a method of treating a patient having rheumatoid arthritis, comprising administering to said patient a therapeutically effective dose of a TLR7/8 inhibitor or a pharmaceutically acceptable salt thereof, in combination with a therapeutically effective dose of a TNFα inhibitor, and in combination with one or more addition third agents. Examples of suitable third agents include corticosteroids, such as prednisone; rolipram, calphostin, cytokine-suppressive anti-inflammatory drugs (CSAIDs), Interleukin-10, glucocorticoids, salicylates, nitric oxide, and other immunosuppressants; nuclear translocation inhibitors, such as deoxyspergualin (DSG); anti-inflammatory drugs such as sulfasalazine; nonsteroidal anti-inflammatory drugs (NSAIDs) such as ibuprofen, celecoxib and rofecoxib; steroids such as dexamethasone; antiproliferative agents such as methotrexate, leflunomide, FK506 (tacrolimus, PROGRAF®); anti-malarials such as hydroxychloroquine; cytotoxic drugs such as azathiprine and cyclophosphamide; and rapamycin (sirolimus or RAPAMUNE®) or derivatives thereof. The above third agents, when employed in combination with the combinations of Compound (I) and the TNFα inhibitor, may be used, for example, in those amounts indicated in the Physicians' Desk Reference (PDR) or as otherwise determined by one of ordinary skill in the art. In the methods of the present invention, the one or more third agents may be administered prior to, simultaneously with, or following the administration of Compound (I) or the second agent.

Preparation of Mouse Enbrel (mEnbrel)

The fully mouse version of Enbrel® was designed with mouse TNFR1B (Ref Seq NP_035740) and a mouse IgG2A isotype (MuTNFR1B(V23-G258)-muIgG2A). It was expressed from stably transfected Chinese Hamster Ovary (CHO) cells with an osteonectin signal sequence. The extracellular domain (ECD) region of muTNFR1B used was residues Val-23 through Gly-258. The ECD was fused directly to the amino terminus of the upper hinge region of mouse heavy chain IgG2A, by analogy to the human Enbrel design.

The mouse Enbrel was expressed in CHO cells in bioreactors at the 90 L scale and was harvested at day 13. It was captured by Protein A (mAb Select), washed with both pH 7.2 phosphate and pH 6.5 acetate buffers, eluted with 50 mM acetic acid, and buffer exchanged into phosphate buffer pH 6.8. The final concentration was 3.1 mg/mL based on an a calculated extinction coefficient of 1.06 mL/(mg*cm). mEnbrel was found to be >97% homogeneous with only 3% high molecular weight by analytical SEC and endotoxin was determined to be 0.035 EU/mg. The material was frozen at −80° C. until use.

### Rheumatoid Arthritis Study in Mice

### Materials and Methods

All the animal experimental procedures were reviewed and approved by the Institutional Animal Ethics Committee (IAEC) and conducted in accordance with procedures set by the Committee For The Purpose of Control and Supervision on Experiments on Animals (CPCSEA). Mice were group housed in Syngene Laboratory Animal Research Facility (SLAR, Bangalore India; AAALAC accredited), and maintained under normal 12 h light/12 h dark cycle with ad libitum access to food and water. At the end of the studies, animals were euthanized by CO₂ asphyxiation for plasma and tissue collection.

### Collagen-Induced Arthritis in Mice

Male DBA/1 mice (9-11 weeks of age, Harlan) were primed with bovine type II collagen (Chondrex #20021) in adjuvant (Sigma adjuvant system, Sigma Aldrich #S6322) at the base of tail on day 1 and on day 21. Mice were randomized into 7 groups based on body weight and assigned as either vehicle (10% ethanol; 45% PEG 300; 5% pluronic F-68; 40% 20 mM citrate buffer); Compound (I) at 0.25 and 2.5 mg/kg or mEnbrel (mouse Enbrel) at 10 mg/kg or combination of Compound (I) with mEnbrel at 0.25+10 mg/kg and 2.5+10 mg/kg or mCTLA4 (mouse CTLA4-Ig) (as a reference compound) at 3 mg/kg dose level. Compound (I) was administered from day 21 by oral gavage once daily whereas mEnbrel and mCTLA4 were administered from day of primary immunization, twice per week by intraperitoneal injection. Disease activity was monitored and scored twice per week using standard criteria (0: normal; 1: mild, but definite redness and swelling of the ankle or wrist, or apparent redness and swelling limited to individual digits regardless of number of affected digits; 2: moderate redness and swelling of ankle or wrist; 3: severe redness and swelling of the entire paw including digits; 4: maximally inflamed limb with involvement of multiple joints). Prior to termination of the experiment, mice were bled at various time points post dose (1 h, 3 h, 7 h, 24 h) to capture the complete pharmacokinetic profile of the Compound (I). Furthermore, at the time of termination, serum and plasma samples were collected to measure IL-6 and anti-collagen antibody titer respectively. Paws were collected for histology analysis.

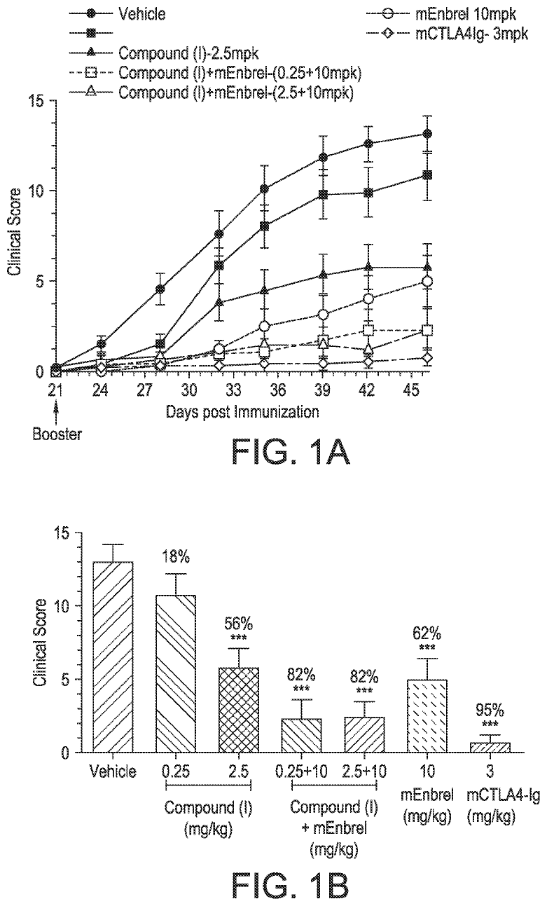

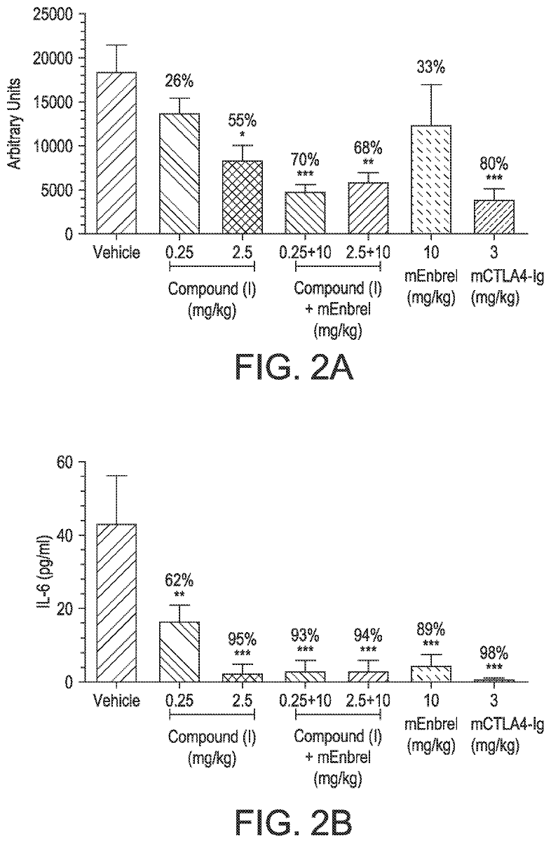

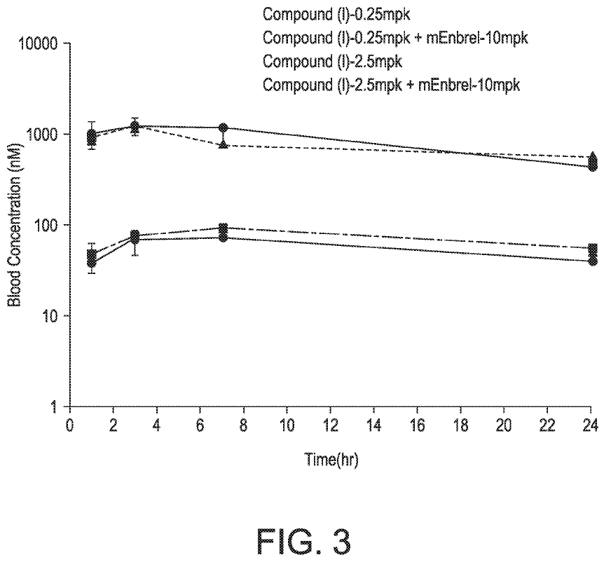

Compound (I) was tested in the semi-therapeutic mode of treatment in mouse collagen-induced arthritis model. Dosing initiated after the antigen boost (from day 21) and continued up to day 45. As shown in FIG. 1A, Compound (I) inhibited clinical signs of disease as early as 7 days post dosing (FIGS. 3.5-1 A). Significant dose dependent suppression of the arthritic score was seen at the termination of the study (FIG. 1B) with dose dependent reduction in plasma IL-6 and serum anti-collagen-antibody titer (FIGS. 2A and 2B).

Compound (I) was tested in combination with the TNFα blocking agent mEnbrel in the collagen-induced arthritis model where dosing of Compound (I) was initiated after the antigen boost whereas mEnbrel was administered from the day of primary immunization. As shown in FIG. 1, the combination of IC₉₀ dose (0.25 mg/kg) and with a fixed dose of mEnbrel (10 mg/kg) resulted in greater suppression of clinical scores when compared to either treatment alone. The 0.25 mg/kg dose of Compound (I) gave equal combination benefit with mEnbrel as the 10-fold higher dose, indicating that IC₉₀ coverage at trough provided robust and maximal combination efficacy with TNFα blockade by mEnbrel. The improvement in efficacy observed in the combination was greater than the additive effect of each treatment alone. Specifically, the 0.25 mg/kg Compound (I) group showed a disease score of 82% of control and the mEnbrel dose group showed a disease score that was 56% of control. If the two drugs were additive the expected value is 31% of control (0.82·0.56=0.31). However the combination showed a disease score that was only 18% of control, indicating that there the combination provided benefit that was substantially greater than would be expected from an additive interaction. The increased efficacy was apparent prior to study termination (FIG. 1A) and was significant at the end of the study (FIG. 1B). The enhanced activity was also reflected in anti-collagen antibody titer (FIG. 2A).

As illustrated in FIG. 3, Compound (I) showed a dose dependent increase in whole blood drug concentration in this study. The whole blood concentration of Compound (I) was not altered in the presence of mEnbrel. The results of this study indicated that the increase in efficacy of the combination was not due to an increase in whole blood concentration of Compound (I).

FIG. 1: Inhibition of arthritic index by Compound (I) alone and in combination with mEnbrel. Mice were treated with respective treatment. During the entire study course (A) and at the time of termination (B) disease was assessed by measuring the clinical score (arthritic index). Data are from one experiment with 10 mice per group. ***P<0.0001 versus vehicle by one-way ANOVA with a Dunnett test.

FIG. 2: Inhibition of circulating markers by Compound (I) alone and in combination with mEnbrel. Mice were treated with respective treatment. At the time of termination (A) serum anti-collagen antibody titer and (B) plasma IL-6 was assessed. Data are from one experiment with 10 mice per group. *P<0.05, **P<0.01, ***P<0.0001 versus vehicle by one-way ANOVA with a Dunnett test.

FIG. 3: Pharmacokinetic analysis of Compound (I) in CIA model of arthritis. Mice were dosed orally for 25 days with Compound (I). Following 19 days of dosing, whole blood was drawn at different time points and DBS drug concentrations were measured by LCMS. Data are from one experiment where drug levels were measured in samples taken at the indicated times from 3 mice per group out of the 10 mice per group dosed in the experiment. Data represent the mean drug concentrations in nM.

MouseTNFR1B(V23-G258)-muIgG2A. Osteonectin signal sequence underlined. Mouse IgG2a in bold.

| (SEQ ID NO: 1) | |
|---|---|
| 1 | MRAWIFFLLC LAGRALAVPA QVVLTPYKPE PGYECQISQE YYDRKAQMCC |
| 51 | AKCPPGQYVK HFCNKTSDTV CADCEASMYT QVWNQFRTCL SCSSSCTTDQ |
| 101 | VEIRACTKQQ NRVCACEAGR YCALKTHSGS CROCMRLSKC GPGFGVASSR |
| 151 | APNGNVLCKA CAPGTFSDTT SSTDVCRPHR ICSILAIPGN ASTDAVCAPE |
| 201 | SPTLSAIPRT LYVSQPEPTR SQPLDQEPGP SQTPSILTSL GSTPIIEQST |
| 251 | KGGEPRGPTI KPCPPCKCPA PNLLGGPSVF IFPPKIKDVL MISLSPIVTC |
| 301 | VVVDVSEDDP DVQISWFVNN VEVHTAQTQT HREDYNSTLR VVSALPIQHQ |
| 351 | DWMSGKEFKC KVNNKDLPAP IERTISKPKG SVRAPQVYVL PPPEEEMTKK |
| 401 | QVTLTCMVTD FMPEDIYVEW TNNGKTELNY KNTEPVLDSD GSYFMYSKLR |
| 451 | VEKKNWVERN SYSCSVVHEG LHNHHTTKSF SRTPGK |

What is claimed is:

1. A method of treating rheumatoid arthritis, comprising administering to a patient a therapeutically effective dose of a TLR7/8 inhibitor or a pharmaceutically acceptable salt thereof, in combination with a therapeutically effective dose of a TNFα inhibitor.

2. The method according to claim 1 wherein said TLR7 inhibitor is: 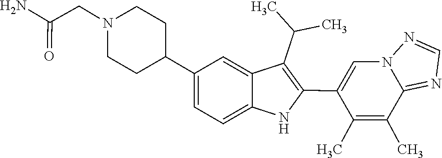 [SMILES: Cc1c(-c2[nH]c3ccc(C4CCN(CC(N)=O)CC4)cc3c2C(C)C)cn2ncnc2c1C]
or a pharmaceutically acceptable salt thereof.

3. The method according to claim 1 wherein said TNFα inhibitor is etanercept or a biosimilar thereof.

4. The method according to claim 1 wherein said TNFα inhibitor is infliximab or a biosimilar thereof.

5. The method according to claim 1 wherein said TNFα inhibitor is certolizumab or a biosimilar thereof.

6. The method according to claim 1 wherein said TNFα inhibitor is golimumab or a biosimilar thereof.

7. The method according to claim 1 wherein said TNFα inhibitor is adalimumab or a biosimilar thereof.

8. The method according to claim 1, wherein said therapeutically effective dose of said TLR7 inhibitor is from 0.1 to 100 mg per day.

9. The method according to claim 1 wherein said TLR7/8 inhibitor and said TNFα inhibitor are administered concurrently.

10. The method according to claim 1 wherein said TLR7/8 inhibitor and said TNFα inhibitor are administered sequentially.

11. The method according to claim 1 wherein said TLR7/8 inhibitor is administered prior to administration of said TNFα inhibitor.

12. The method according to claim 1 wherein said TNFα inhibitor is administered prior to administration of said TLR7/8 inhibitor.

13. A polypeptide comprising amino acid residues 18-486 of SEQ ID NO:1.

14. The polypeptide of claim 13, wherein the polypeptide comprises the amino acid sequence of SEQ ID NO:1.

15. The method according to claim 2 wherein said TNFα inhibitor is etanercept or a biosimilar thereof.

16. The method according to claim 2 wherein said TNFα inhibitor is infliximab or a biosimilar thereof.

17. The method according to claim 2 wherein said TNFα inhibitor is certolizumab or a biosimilar thereof.

18. The method according to claim 2 wherein said TNFα inhibitor is golimumab or a biosimilar thereof.

19. The method according to claim 2 wherein said TNFα inhibitor is adalimumab or a biosimilar thereof.


SEQUENCE LISTING (1 sequence)

SEQ ID NO: 1 - protein, 486 aa, Mus musculus
  SIGNAL (1)..(17): Osteonectin signal sequence
  MISC_FEATURE (18)..(253): Mouse TNFR1B (V23 - G258)
  MISC_FEATURE (254)..(486): Mouse IgG2a
MRAWIFFLLCLAGRALAVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK